(19) United States
(12) Patent Application Publication
(10) Pub. No.: US 2018/0338753 A1
(43) Pub. Date: Nov. 29, 2018

(54) SUTURE ANCHOR SYSTEM WITH REUSABLE HANDLE

(71) Applicant: Smith & Nephew, Inc., Memphis, TN (US)

(72) Inventor: Mark E. Housman, North Attleboro, MA (US)

(73) Assignee: Smith & Nephew, Inc., Memphis, TN (US)

(21) Appl. No.: 15/540,626

(22) PCT Filed: Jan. 5, 2016
(86) PCT No.: PCT/US2016/012137

§ 371 Date: Jun. 29, 2017

Related U.S. Application Data
(60) Provisional application No. 62/100,202, filed on Jan. 6, 2015.

(51) Int. Cl. A61B 17/04

(52) U.S. Cl. CPC A61B 17/0401; A61B 2017/00004; A61B 2017/0409; A61B 2017/0445

(57) ABSTRACT

Disclosed is a single-use suture anchor and shaft cartridge, the cartridge being removably attached to a reusable handle. One or more sutures may be secured to the suture anchor and routed through interior cannulations of both the suture anchor and the shaft. The ends of the sutures may then be passed to the outside of the shaft either through an aperture in the shaft or into a communicating cannulation in the handle assembly. The ends of the suture then may be passed to the outside of the handle either through an opening at the proximal end of the handle or through a sidewall opening in the handle.

### FIELD

The present application relates generally to suture anchor systems for tissue repair and, in particular, to suture anchor systems with reusable handles.

### BACKGROUND

Suture anchors are devices employed in surgical operations. Typically, a suture anchor is loaded with a suture and subsequently implanted within bone. The suture anchor secures a portion of the suture in place with respect to the bone. The suture may be further routed through soft tissue and/or bone for fixation.

An inserter tool is typically used to implant the suture anchor into bone. The inserter tool may include a handle connected to a shaft which is engaged with the suture anchor. By manipulation of the inserter tool, the suture anchor may be positioned in a desired location. Most inserter tools are designed for single use, in which both the handle and the shaft are disposed of after suture anchor insertion.

Since much of the cost of a suture anchor system is attributable to the inserter tool, some fully reusable inserter tools have been developed. However, since these inserter tools typically include both the shaft and the handle, they require greater clinical manipulation to prepare them for use. Use of these inserter tools requires the surgeon to connect the suture to the suture anchor, connect the suture anchor to the inserter tool and appropriately route the suture within the inserter tool, amongst other surgical preparation operations. Such inserter tools also present challenges for sterilization.

### SUMMARY

Described herein are improved, cost-effective and sterile suture anchor systems that allow for preferred suture routing through the shaft and/or the handle. The suture anchor systems of this disclosure include a single-use suture anchor and shaft cartridge, the cartridge being removably attached to a reusable handle. One or more sutures may be secured to the suture anchor and routed through interior cannulations of both the suture anchor and the shaft. The ends of the sutures may then be passed to the outside of the shaft either through an aperture in the shaft or into a communicating cannulation in the handle assembly. In the latter case, the ends of the suture then may be passed to the outside of the handle either through an opening at the proximal end of the handle or through a sidewall opening in the handle.

Examples of the single-use suture anchor and shaft cartridge may include a cartridge with a suture anchor having a proximal end and distal end, a cannulation within the suture anchor extending longitudinally from the proximal end to the distal end; a shaft having a proximal end and distal end, the proximal end of the shaft configured to receive a mating end of a reusable handle, the shaft further including an engagement portion extending from the distal end of the shaft adapted to slidably receive the cannulation of the suture anchor, and a lumen within the shaft extending longitudinally from the proximal end to the distal end of the shaft. The cartridge may also include at least one pre-loaded suture slidably coupled to the suture anchor, with the suture limbs routed through and extending from the lumen of the shaft. In other examples, the shaft may further include an aperture formed in an outer wall of the shaft and in communication with the lumen, the aperture being adjacent to the proximal end of the shaft. The limbs of the at least one suture may extend from the lumen through the aperture.

Examples of the suture anchor system may include the suture anchor and shaft cartridge described above and a handle having a proximal and a distal end, the distal end of the handle adapted to releasably engage the proximal end of the shaft. The limbs of the at least one suture may extend from the lumen through the proximal end of the shaft. The handle may further comprise a cannulation within the handle extending from the proximal end to the distal end of the handle, the handle cannulation adapted to receive the at least one suture extending through the proximal end of the shaft.

Examples of a kit may including one or more single-use, disposable suture anchor and shaft cartridges together with a reusable handle.

The suture anchor system described herein possesses a variety of benefits over both fully-disposable and fully-reusable suture anchor systems. Cost savings are achieved since the handle does not require replacement after each use. Furthermore, the suture anchor and shaft cartridge may be provided to a surgeon with the anchor, suture, and shaft in an assembled configuration. As a result, the surgeon need only attach the anchor and shaft cartridge to the handle to prepare the system for use. After the suture anchor system is inserted at a desired position in the bone, the shaft may be disengaged from the handle and disposed of, while the handle is retained for subsequent use with another anchor and shaft cartridge.

### BRIEF DESCRIPTION OF THE DRAWINGS

The foregoing and other objects, features and advantages will be apparent from the following more particular description of the examples, as illustrated in the accompanying drawings in which like reference characters refer to the same parts throughout the different views. The drawings are not necessarily to scale, emphasis instead being placed upon illustrating the principles of the examples.

FIG. 1A is a side view of a single-use suture anchor and shaft cartridge removably assembled to a reusable handle of this disclosure;

FIG. 1B is a perspective view of the reusable handle of FIG. 1A;

FIG. 2 is an exploded view of the suture anchor and the distal end of the shaft;

FIG. 3A is a cross-section of the proximal end of the shaft assembled to the handle;

FIG. 3B is a side view of the suture anchor and shaft cartridge of FIG. 1A, with suture limbs extending through the shaft;

FIG. 4A-C are further examples of the handle of FIG. 1B; and

FIG. 5 is an example of a kit of this disclosure

### DETAILED DESCRIPTION

Examples of the disclosed suture anchor and shaft cartridge will now be discussed with reference to the figures.

In the description that follows, like components have been given the same reference numerals, regardless of whether they are shown in different examples. To illustrate an example(s) of the present invention in a clear and concise manner, the drawings may not necessarily be to scale and certain features may be shown in somewhat schematic form. Features that are described and/or illustrated with respect to one example may be used in the same way or in a similar way in one or more other examples and/or in combination with or instead of the features of the other examples.

Comprise, include, and/or plural forms of each are open ended and include the listed parts and can include additional parts that are not listed. And/or is open ended and includes one or more of the listed parts and combinations of the listed parts.

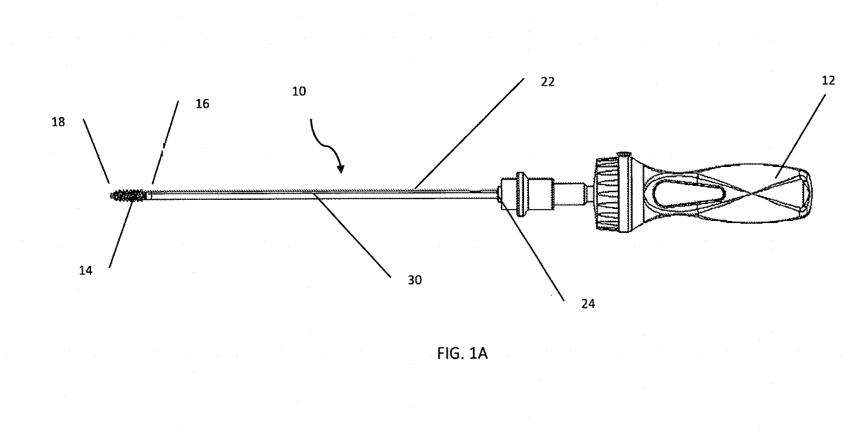

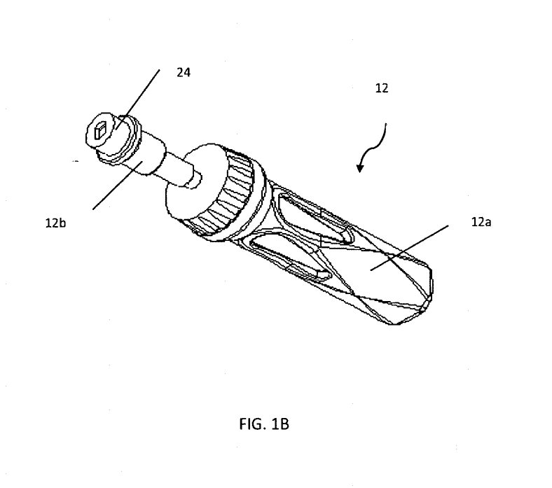

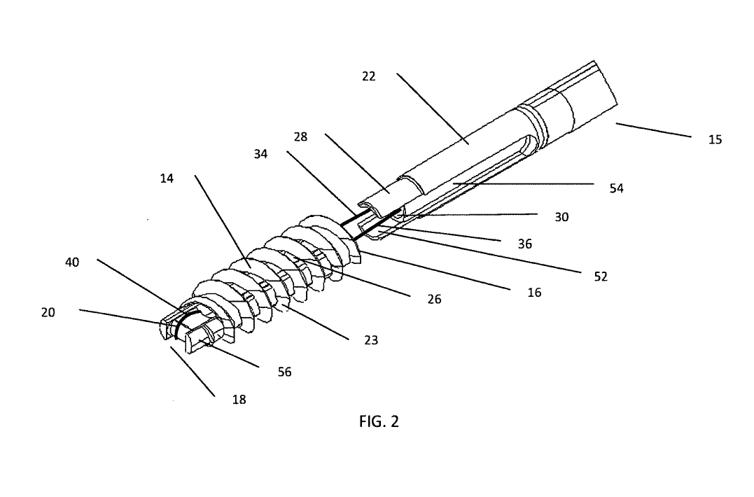

FIG. 1A shows a side view of a single-use suture anchor and shaft cartridge 10 removably assembled to a reusable handle 12 for performing a tissue repair procedure. The suture anchor and shaft cartridge 10 generally includes a suture anchor 14 having a proximal end 16 and distal end 18. The suture anchor and shaft cartridge 10 can include a shaft 22 configured to releasably receive a mating end 24 of the reusable handle 12. The shaft 22 further includes a lumen 30 (shown in more detail in FIG. 2) extending longitudinally through the shaft 22. The shaft 22 may be made from a metal material, such as stainless steel and titanium. However, other metal and non-metal materials that would withstand the forces applied during surgery may be used. The length and diameter of the shaft 22 may vary.

FIG. 1B shows a perspective view of the reusable handle 12. The handle 12 includes a grip 12a and a connector 12b coupled to the grip 12a. The grip 12a of handle 12 can be made from plastic. However, other non-metal and metal materials may also be used. The shape and size of the grip 1.2a may be any shape and size necessary to help facilitate insertion of the suture anchor 14 into bone. The connector 12b is made from a metal material, such as stainless steel or titanium, but may be made from other metal and non-metal materials that are strong enough to withstand the forces applied during surgery. The connector 12b is press-fit to the grip 12a, but may be coupled to the grip 12a in any other manner known to those of skill in the art. The size and shape of the connector 12b may be any size and shape necessary to help facilitate insertion of the suture anchor 14 into bone. A distal end of the connector 12b includes mating end 24. The handle 12 may also be comprised of materials capable of undergoing sterilization. It is also contemplated by this disclosure that the reusable handle 12 may also include one or more customizable features that would not be cost-effective to introduce into a single-use handle. Examples may include, but are not limited to, ergonomic customizations, such as adapting the handle for a specific surgeon's grip, and/or use of more advanced materials in construction (e.g., shape memory gels/foams, etc.).

FIG. 2 is an exploded view of the suture anchor 14 and a distal end 15 of the shaft 22. In FIG. 2, a majority of the suture anchor 14 includes screw threads 23 in the form of an open helical coil, i.e., a connected series of continuous regularly spaced turns extending in a helical or spiral form substantially from a proximal end 16 to the distal end 18 with apertures being defined by the space between the turns of the coil. The open helical coil defines a cannulation 26 extending longitudinally from the proximal end 16 to the distal end 18 of the suture anchor 14 and communicating with the region exterior to the open helical coil through the spacing between the turns of the screw threads 23. The size, shape, spacing and number of screw threads 23 may vary. The suture anchor 14 can be made from a polymer or bioreabsorbable material via a molding method. However, other material which would allow the suture anchor 14 to withstand forces during surgery and other methods of making may be used. The distal end 18 of the suture anchor 14 also includes a suture bridge 20 that extends a partial length of the suture anchor 14. The size, shape and thickness of the suture bridge 20 may vary. It is also contemplated by this disclosure that other configurations of suture anchor 14 may be used.

The distal end 15 of the shaft 22 includes an engagement portion 28 adapted to slidably receive the cannulation 26 of the suture anchor 14 thereon. The engagement portion 28 may slidably receive the cannulation 26 in a variety of ways. For instance, the engagement portion 28 may include a slot 52 and grooves 54 extending from the slot 52 on each side of the shaft 22. In turn, longitudinally extending runners 56 may extend from the suture bridge 20 of the suture anchor 14 and along the interior of the screw threads 23. When the engagement portion 28 of the shaft 22 and the cannulation 26 of the suture anchor 14 are coupled, the suture bridge 20 is housed within the slot 52 and the runners 56 are housed within the grooves 54. Other configurations and couplings between the engagement portion 28 of the shaft 22 and the cannulation 26 of the suture anchor 14 can be found in U.S. Publication No. 2015/0327984, the contents of which are incorporated herein by reference. As further shown in FIG. 2, a suture 40 can be secured to the suture anchor 14 with a portion of the suture 40 being routed around the suture bridge 20. In this configuration, suture limbs 34, 36 are routed through and extend out of the cannulation 26 and into the lumen 30 of the shaft 22.

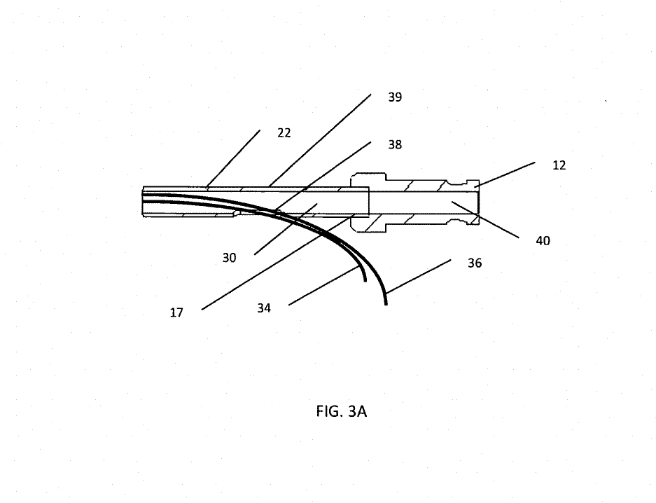

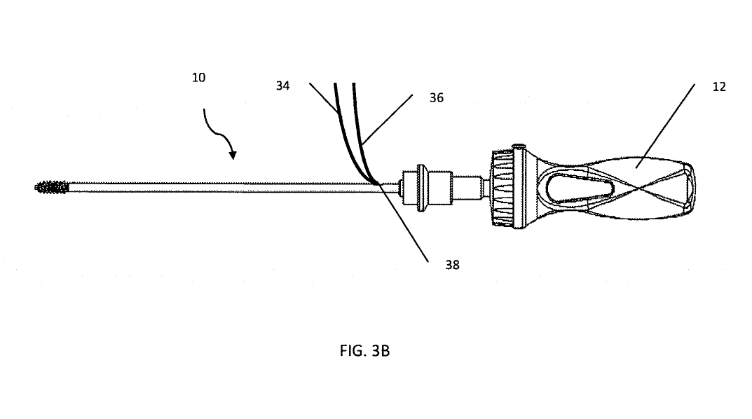

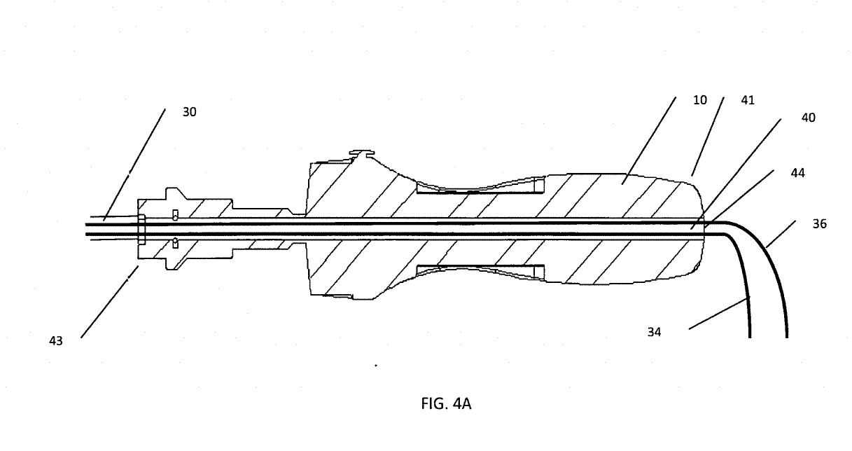

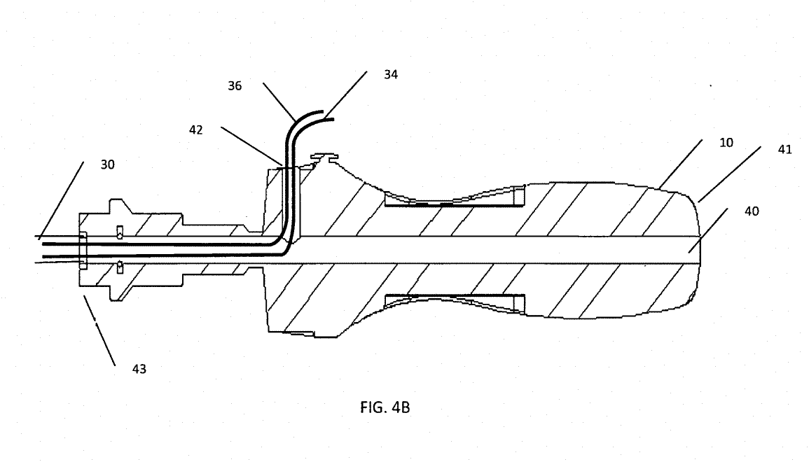

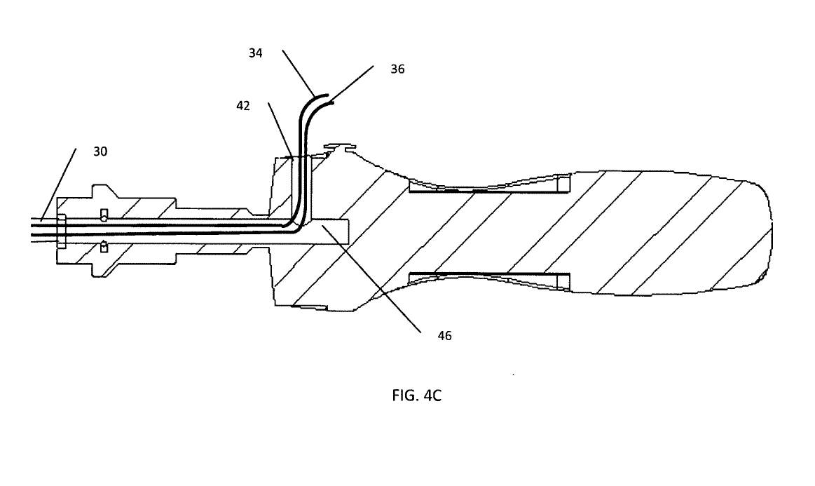

FIG. 3A illustrates a cross-section of the proximal end 17 of the shaft 22 assembled to the handle 12. An aperture 38 can be formed in an outer wall 39 of the shaft 22 at the proximal end 17 in communication with the lumen 30. Alternatively, the aperture 38 may be positioned closer to the distal end 15 of the shaft 22 (not shown). FIG. 3A also shows both the suture limbs 34, 36 routed through the lumen 30 and extending through the aperture 38 to an area outside of the shaft 22. Alternatively, the suture limbs 34, 36 may also extend from the lumen 30 at the proximal end 17 of the shaft 22 into a communicating cannulation 40 in the handle assembly 12, as shown in FIG. 4A-C below. FIG. 3B shows a side view of the suture anchor and shaft cartridge 10 assembled to the reusable handle 12 of FIG. 1, with the suture limbs 34, 36 extending through the aperture 38.

FIG. 4A shows the handle 12 with a longitudinal cannulation 40 extending from the proximal end 41 of the handle 12 to the distal end 43 of the handle 12, in communication with the lumen 30 of the shaft 22. In this example, the suture limbs 34, 36 extending through lumen 30 may further extend through the longitudinal cannulation 40 and out of the handle 12 through an opening 44 at the proximal end 41 of the handle 12. As shown in FIG. 4B, the longitudinal cannulation 40 may extend from the distal end 43 to the proximal end 41 of the handle 12, but the suture limbs 34, 36 instead extend through a sidewall opening 42 to the exterior of the handle assembly 12. FIG. 4C shows a handle assembly 12 with a partial cannulation 46 only, the suture limbs 34, 36 again extending through a sidewall opening 42 of the handle assembly 12.

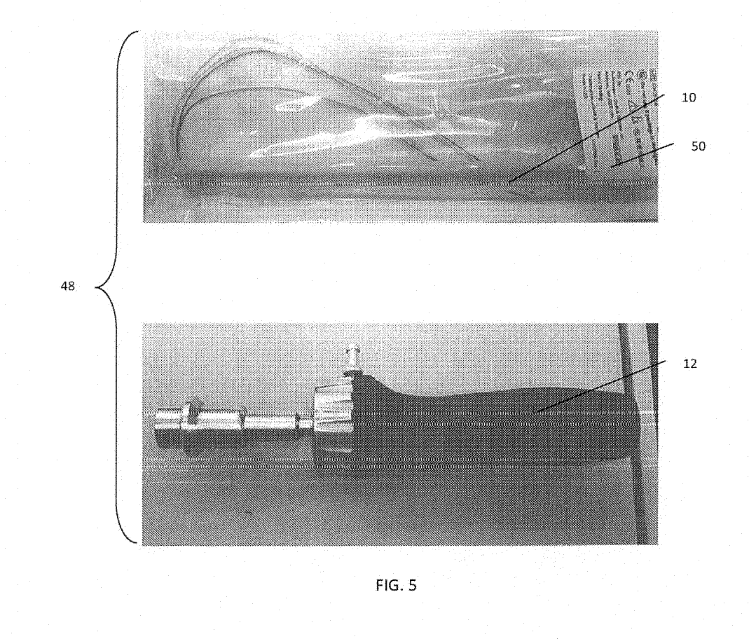

As shown in FIG. 5, a kit 48 may be provided including one or more single-use, sterile, disposable suture anchor and shaft cartridges 10 together with a reusable handle 12. The kit may also include a package insert or label 50.

One skilled in the art will realize the invention may be embodied in other specific forms without departing from the spirit or essential characteristics thereof. The foregoing examples are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

What is claimed is:

1. A single-use suture anchor and shaft cartridge for performing a tissue repair procedure, the cartridge comprising:
a suture anchor having a proximal end and distal end, a longitudinal cannulation within the suture anchor extending from the proximal end to the distal end of the suture anchor;
a shaft having a proximal end and distal end, the proximal end of the shaft configured to receive a mating end of a reusable handle, the shaft further comprising:
an engagement portion extending from the distal end of the shaft, the engagement portion adapted to slidably receive the cannulation of the suture anchor thereon; and
a lumen within the shaft extending longitudinally from the proximal end to the distal end of the shaft; and
at least one pre-loaded suture slidably coupled to the suture anchor, the at least one pre-loaded suture including suture limbs routed through and extending from the lumen of the shaft.

2. The suture anchor and shaft cartridge of claim 1, wherein the suture limbs extending from the lumen of the shaft comprises the suture limbs extending through an aperture formed in an outer wall of the shaft in communication with the lumen.

3. The suture anchor and shaft cartridge of claim 2, wherein the aperture is adjacent to the proximal end of the shaft.

4. The suture anchor and shaft cartridge of claim 2, wherein the aperture is adjacent to the distal end of the shaft.

5. The suture anchor and shaft cartridge of claim 1, wherein the suture anchor comprises a suture bridge.

6. The suture anchor and shaft cartridge of claim 5, wherein a portion of the suture is routed around the suture bridge.

7. The suture anchor and shaft cartridge of claim 1, wherein the suture anchor is comprised of a polymer.

8. The suture anchor and shaft cartridge of claim 1, wherein the suture anchor is comprised of a bioreabsorbable material.

9. The suture anchor and shaft cartridge of claim 1, wherein the shaft is comprised of metal.

10. The suture anchor and shaft cartridge of claim 1, wherein the suture anchor is an open helical coil anchor.

11. A suture anchor system for performing a tissue repair procedure, the suture anchor system comprising:
a single-use suture anchor and shaft cartridge, the cartridge comprising:
a suture anchor having a proximal end and distal end, a longitudinal cannulation within the suture anchor extending from the proximal end to the distal end of the suture anchor;
a shaft having a proximal end and distal end, the proximal end of the shaft configured to receive a mating end of a reusable handle, the shaft further comprising:
an engagement portion extending from the distal end of the shaft, the engagement portion adapted to slidably receive the cannulation of the suture anchor thereon; and
a lumen within the shaft extending longitudinally from the proximal end to the distal end of the shaft; and
at least one pre-loaded suture slidably coupled to the suture anchor, the at least one pre-loaded suture including suture limbs routed through and extending from the lumen of the shaft; and
a handle having a proximal and a distal end, the distal end of the handle adapted to releasably engage the proximal end of the shaft.

12. The suture anchor system of claim 11, wherein the handle further comprises a cannulation within the handle.

13. The suture anchor system of claim 12, wherein the cannulation extends from a proximal to a distal end of the handle.

14. The suture anchor system of claim 12, wherein the cannulation extends from a distal end of the handle to an area proximal to the proximal end of the handle.

15. The suture anchor system of claim 12, wherein the suture limbs extending from the lumen of the shaft comprises the suture limbs extending from the lumen of the shaft into the cannulation within the handle.

16. The suture anchor system of claim 11, wherein the handle is comprised of a material capable of undergoing sterilization.

17. The suture anchor system of claim 11, wherein the engagement portion comprises a slot and at least one groove extending from the slot on a side of the shaft, the at least one groove configured to receive at least one longitudinally extending runner of the suture anchor.

18. A kit for performing a tissue repair procedure, the kit comprising:
a single-use suture anchor and shaft cartridge, the cartridge comprising:
a suture anchor having a proximal end and distal end, a longitudinal cannulation within the suture anchor extending from the proximal end to the distal end of the suture anchor;
a shaft having a proximal end and distal end, the proximal end of the shaft configured to receive a mating end of a reusable handle, the shaft further comprising:
an engagement portion extending from the distal end of the shaft, the outer surface adapted to slidably receive the cannulation of the suture anchor thereon; and
a lumen within the shaft extending longitudinally from the proximal end to the distal end of the shaft; and
at least one pre-loaded suture slidably coupled to the suture anchor, the at least one pre-loaded suture including suture limbs routed through and extending from the lumen of the shaft;
a reusable handle; and
a package insert or label.

19. The kit of claim 18, wherein the reusable handle has a proximal and a distal end, the distal end of the handle adapted to releasably engage the proximal end of the shaft.

20. The kit of claim 18, wherein the reusable handle is comprised of a material capable of undergoing sterilization.
